(19) United States
(12) Patent Application Publication
(10) Pub. No.: US 2019/0255331 A1
(43) Pub. Date: Aug. 22, 2019

(54) HIGH FREQUENCY MODULATION OF SACRAL NERVES TO ADDRESS PELVIC PAIN AND GASTROUROGENITAL CONDITIONS, AND ASSOCIATED SYSTEMS AND METHODS

(71) Applicant: Nevro Corp., Redwood City, CA (US)

(72) Inventor: Jeyakumar Subbaroyan, Floral Park, NY (US)

(21) Appl. No.: 16/276,536

(22) Filed: Feb. 14, 2019

Related U.S. Application Data
(60) Provisional application No. 62/631,751, filed on Feb. 17, 2018.

(51) Int. Cl. A61N 1/36; A61N 1/05

(52) U.S. Cl. CPC A61N 1/36132; A61N 1/0553; A61N 1/36071; A61N 1/36007; A61N 1/36062

(57) ABSTRACT

Systems and methods for treating a patient having pelvic pain and one or more gastrourogenital conditions using electrical stimulation are disclosed. A representative method for treating a patient includes directing an electrical therapy signal to the patient's sacral region, via an implantable signal delivery device, to reduce or eliminate pelvic pain and/or one or more gastrourogenital conditions in the patient, wherein the electrical signal has a frequency in a frequency range of from 1.2 kHz to 100 kHz and does not produce paresthesia in the patient.

### CROSS-REFERENCE TO RELATED APPLICATION

The present application claims priority to pending U.S. Provisional Application No. 62/631,751, filed on Feb. 17, 2018, and are incorporated herein by reference.

### TECHNICAL FIELD

The present technology is directed generally to methods and systems for treating pain and one or more gastrourogenital conditions of a patient by applying electrical stimulation to a target neural population located within one or more of the patient's sacral nerves.

### BACKGROUND

Neurological stimulators have been developed to treat pain, movement disorders, functional disorders, spasticity, cancer, cardiac disorders, and various other medical conditions. Implantable neurological stimulation systems generally have an implantable signal generator and one or more leads that deliver electrical pulses to neurological tissue or muscle tissue. For example, several neurological stimulation systems for spinal cord stimulation (SCS) have cylindrical leads that include a lead body with a circular cross-sectional shape and one or more conductive rings (i.e., contacts) spaced apart from each other at the distal end of the lead body. The conductive rings operate as individual electrodes and, in many cases, the SCS leads are implanted percutaneously through a needle inserted into the epidural space, with or without the assistance of a stylet.

Once implanted, the signal generator applies electrical pulses to the electrodes, which in turn modify the function of the patient's nervous system, such as by altering the patient's responsiveness to sensory stimuli and/or altering the patient's motor-circuit output. In SCS therapy for the treatment of pain, the signal generator applies electrical pulses to the spinal cord via the electrodes. In conventional SCS therapy, electrical pulses are used to generate sensations (known as paresthesia) that mask or otherwise alter the patient's sensation of pain.

Aspects of the present disclosure are directed to methods and systems that make use of, employ, rely on and/or otherwise use or incorporate aspects of the interaction between electrical therapy and the patients to whom the therapy is applied.

### BRIEF DESCRIPTION OF THE DRAWINGS

FIG. 1A is a partially schematic illustration of an implantable spinal cord modulation system positioned at the spine to deliver therapeutic signals in accordance with some embodiments of the present technology.

FIG. 1B is a partially schematic, cross-sectional illustration of a patient's spine, illustrating representative locations for implanted lead bodies in accordance with some embodiments of the present technology.

FIG. 2 is a partially schematic illustration of a patient's sacral region, illustrating representative locations for implanted lead bodies in accordance with some embodiments of the present technology.

FIG. 3 is a chart summarizing outcomes of an OAB Feasibility Study in accordance with some embodiments of the present technology.

FIG. 4 is a chart illustrating visual analog scale (VAS) scores for four patients who reported having pain during the entry criteria evaluation and baseline characteristic measurement, enrolled in the OAB Feasibility Study in accordance with some embodiments of the present technology.

FIG. 5 is a chart illustrating an average number of voids within a 24-hour period for patients enrolled in the OAB Feasibility Study in accordance with some embodiments of the present technology.

FIG. 6 is a chart illustrating an average number of leaks within a 24-hour period for patients enrolled in the OAB Feasibility Study in accordance with some embodiments of the present technology.

FIG. 7 is a chart illustrating an average number of times a patient's bladder failed to completely empty within a 24-hour period for patients enrolled in the OAB Feasibility Study in accordance with some embodiments of the present technology.

FIG. 8 is a chart illustrating changes in fecal incontinence reported by patients enrolled in the OAB Feasibility Study between baseline and end of trial (EoT) in accordance with some embodiments of the present technology.

### DETAILED DESCRIPTION

### 1.0 Introduction

The present technology is directed generally to spinal cord modulation and associated systems and methods for treating pain (e.g., pelvic pain) and a gastrourogenital condition via waveforms with high frequency elements or components (e.g., portions having high fundamental frequencies). “Gastrourogenital condition” as used herein refers to a condition, disease, disorder, deformity, infection, or pathological and/or physiological abnormality that affects a patient's urinary tract (e.g., kidneys, bladder, ureters, urethra); reproductive organs; digestive tract (e.g., stomach, large intestine, small intestine, colon); rectum; and/or a muscle or nerve related to the patient's urinary tract, reproductive organs, digestive tract, rectum, or colon. Such gastrourogenital conditions can include overactive bladder, urinary urge (e.g., increased urge to urinate), nocturia (e.g, wakening at night due to an urge to urinate), incontinence (e.g. urinary and/or fecal which includes leak), increased frequency of urination (e.g., greater than eight voids per day), urinary retention (e.g., incomplete emptying), detrusor muscle overactivity and/or instability (all of the above stated in both idiopathic and neurogenic patients), hysterectomy, endometriosis, interstitial cystitis/painful bladder syndrome, pudendal neuralgia, pelvic, and/or abdominal pain and/or sexual dysfunction.

The systems and methods described herein may treat pelvic pain and one or more gastrourogenital conditions generally with reduced or eliminated side effects. Such side effects can include unwanted motor stimulation or blocking, and/or interference with sensory functions other than the targeted pelvic pain and gastrourogenital condition. Some embodiments continue to provide pain relief and relief from a gastrourogenital condition for at least some period of time after the modulation signals have ceased. As described further below, the modulation signals include sacral nerve modulation (SNM) signals, and/or peripheral nerve modulation signals which can be delivered to the patient's tibial nerve, sciatic nerve, pudendal nerve, dorsal penile nerve, and/or clitoral nerve. Although some embodiments are described below with reference to modulating the dorsal column, dorsal horn, dorsal root, dorsal root entry zone, other particular regions of the spinal column, and/or the sacral nerve(s) to control pelvic pain and treat a gastrourogenital condition, the modulation may, in some instances, be directed to other neurological structures, such as peripheral nerves, and/or target neural populations of other neurological tissues, organs, and/or tissues.

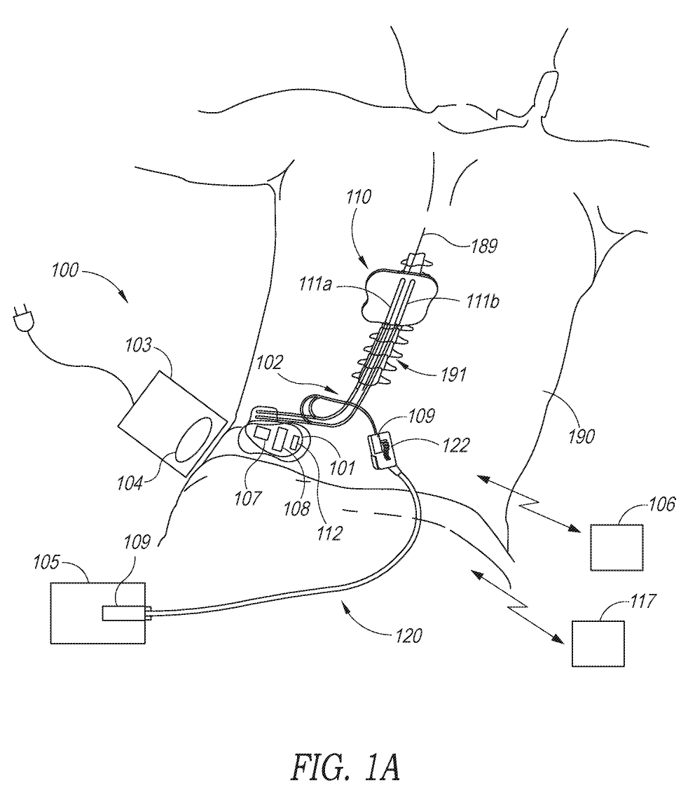

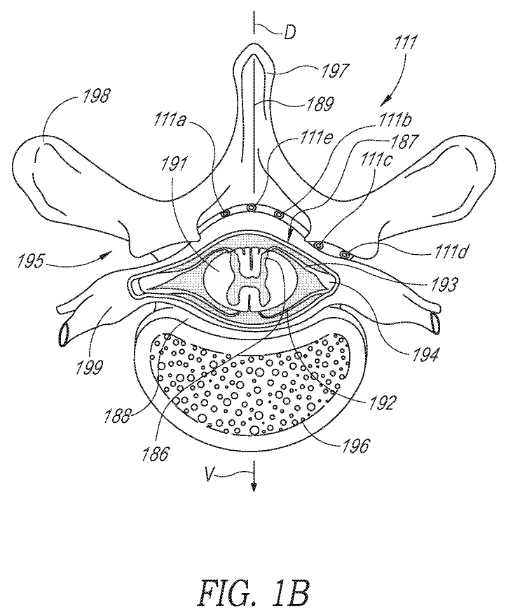

Specific details of some embodiments of the present technology are described below with reference to methods for modulating one or more target neural populations within the patient's sacral region (e.g., sacral nerves) or other sites of a patient and associated implantable structures for providing the modulation. Some embodiments can have configurations, components or procedures different than those which are described herein, and other embodiments may eliminate particular components or procedures. A person of ordinary skill in the relevant art, therefore, will understand that the present disclosure may include some embodiments with additional elements, and/or may include some embodiments without several of the features shown and described below with reference to FIGS. 1A-8.

In general terms, the present technology is directed to producing a therapeutic effect that includes reducing or eliminating pelvic pain and reducing or eliminating a gastrourogenital condition (or one or more symptoms thereof) in the patient. The therapeutic effect can be produced by inhibiting, suppressing, downregulating, blocking, preventing, and/or otherwise modulating the activity of the affected and/or target neural population, such as in the sacral region. In some embodiments of the presently disclosed techniques, therapy-induced paresthesia is not a prerequisite to achieving pain reduction, unlike standard SCS or SNM techniques. It is also expected that the techniques described below with reference to FIGS. 1A-8 can produce more effective, more robust, less complicated and/or otherwise more desirable results than can existing and/or non-existing stimulation therapies. In particular, these techniques can produce results that (1) reduce or eliminate pelvic pain and reduce or eliminate a gastrourogenital condition, and (2) persist after the modulation signal ceases. Accordingly, these techniques can use less power than existing techniques because they need not require delivering modulation signals continuously to obtain a beneficial effect.

Provided herein are various embodiments of neuromodulation systems, methods, and therapies for the reduction and/or treatment of pelvic pain and gastrourogenital conditions. Unless otherwise specified, the specific embodiments discussed are not to be construed as limitations on the scope of the disclosed technology. It will be apparent to one skilled in the art that various equivalents, changes, and modifications may be made without departing from the scope of the disclosed technology, and it is understood that such equivalent embodiments are to be included herein.

### 2.0 System Characteristics

FIG. 1A schematically illustrates a representative patient therapy system 100 for providing relief from chronic pain and a gastrourogenital condition, arranged relative to the general anatomy of a patient's spinal column 191. The system 100 can include a signal generator 101 (e.g., an implanted or implantable pulse generator or IPG), which may be implanted subcutaneously within a patient 190 and coupled to one or more signal delivery elements or devices 110. The signal delivery elements or devices 110 may be implanted within the patient 190, at or off the patient's spinal cord midline 189. The signal delivery elements 110 carry features for delivering therapy to the patient 190 after implantation. The signal generator 101 can be connected directly to the signal delivery devices 110, or it can be coupled to the signal delivery devices 110 via a signal link, e.g., a lead extension 102. In some embodiments, the signal delivery devices 110 can include one or more elongated lead(s) or lead body or bodies 111 (identified individually as a first lead 111a and a second lead 111b). As used herein, the terms signal delivery device, lead, and/or lead body include any of a number of suitable substrates and/or supporting members that carry electrodes/devices for providing therapy signals to the patient 190. For example, the lead or leads 111 can include one or more electrodes or electrical contacts that direct electrical signals into the patient's tissue, e.g., to provide for therapeutic relief. In some embodiments, the signal delivery elements 110 can include structures other than a lead body (e.g., a paddle) that also direct electrical signals and/or other types of signals to the patient 190, e.g., as disclosed in U.S. Provisional Patent Application No. 62/469,430, which is incorporated herein by reference in its entirety.

In some embodiments, one signal delivery device may be implanted on one side of the spinal cord midline 189, and a second signal delivery device may be implanted on the other side of the spinal cord midline 189. For example, the first and second leads 111a, 111b shown in FIG. 1A may be positioned just off the spinal cord midline 189 (e.g., about 1 mm offset) in opposing lateral directions so that the two leads 111a and 111b are spaced apart from each other by about 2 mm. In some embodiments, the leads 111 may be implanted at a vertebral level ranging from, for example, about T8 to about T12. In some embodiments, one or more signal delivery devices can be implanted at other vertebral levels, e.g., as disclosed in U.S. Patent Application Publication No. 2013/0066411 which is incorporated herein by reference in its entirety. For example, the one or more signal delivery devices can be implanted using methods and at locations suitable for DBS, PNS, sacral nerve stimulation, and/or other types of stimulation involving implantable leads.

The signal generator 101 can transmit signals (e.g., electrical signals) to the signal delivery elements 110 that excite and/or suppress target nerves. As used herein, and unless otherwise noted, the terms “modulate,” “modulation,” “stimulate” and “stimulation” refer generally to signals that have either type of the foregoing effects on the target nerves. Accordingly, a spinal cord “stimulator” can have an inhibitory effect on certain neural populations. The signal generator 101 can include a machine-readable (e.g., computer-readable) or controller-readable medium containing instructions for generating and transmitting suitable therapy signals. The signal generator 101 and/or other elements of the system 100 can include one or more processor(s) 107, memory unit(s) 108, and/or input/output device(s) 112. Accordingly, the process of providing modulation signals, providing guidance information for positioning the signal delivery devices 110, establishing battery charging and/or discharging parameters, and/or executing other associated functions can be performed by computer-executable instructions contained by, on or in computer-readable media located at the pulse generator 101 and/or other system components. Further, the pulse generator 101 and/or other system components may include dedicated hardware, firmware, and/or software for executing computer-executable instructions that, when executed, perform any one or more methods, processes, and/or sub-processes described in the references incorporated herein by reference, in their entirety. The dedicated hardware, firmware, and/or software also serve as “means for” performing the methods, processes, and/or sub-processes described herein. The signal generator 101 can also include multiple portions, elements, and/or subsystems (e.g., for directing signals in accordance with multiple signal delivery parameters) carried in a single housing, as shown in FIG. 1A, or in multiple housings.

The signal generator 101 can also receive and respond to an input signal received from one or more sources. The input signals can direct or influence the manner in which the therapy, charging, and/or process instructions are selected, executed, updated, and/or otherwise performed. The input signals can be received from one or more sensors (e.g., an input device 112 shown schematically in FIG. 1A for purposes of illustration) that are carried by the signal generator 101 and/or distributed outside the signal generator 101 (e.g., at other patient locations) while still communicating with the signal generator 101. The sensors and/or other input devices 112 can provide inputs that depend on or reflect patient state (e.g., patient position, patient posture, and/or patient activity level), and/or inputs that are patient-independent (e.g., time). Still further details are included in U.S. Pat. No. 8,355,797, incorporated herein by reference in its entirety.

In some embodiments, the signal generator 101 and/or signal delivery devices 110 can obtain power to generate the therapy signals from an external power source 103. For example, the external power source 103 can bypass an implanted signal generator and generate a therapy signal directly at the signal delivery devices 110 (or via signal relay components). The external power source 103 can transmit power to the implanted signal generator 101 and/or directly to the signal delivery devices 110 using electromagnetic induction (e.g., RF signals). For example, the external power source 103 can include an external coil 104 that communicates with a corresponding internal coil (not shown) within the implantable signal generator 101, signal delivery devices 110, and/or a power relay component (not shown). The external power source 103 can be portable for ease of use.

In some embodiments, the signal generator 101 can obtain the power to generate therapy signals from an internal power source in addition to, or in lieu of, the external power source 103. For example, the implanted signal generator 101 can include a non-rechargeable battery or a rechargeable battery to provide such power. When the internal power source includes a rechargeable battery, the external power source 103 can be used to recharge the battery. The external power source 103 can, in turn, be recharged from a suitable power source (e.g., conventional wall power).

During at least some procedures, an external stimulator or trial modulator 105 can be coupled to the signal delivery elements 110 during an initial procedure prior to implanting the signal generator 101. For example, a practitioner (e.g., a physician and/or a company representative) can use the trial modulator 105 to vary the modulation parameters provided to the signal delivery elements 110 in real time and select optimal or particularly efficacious parameters. These parameters can include the location from which the electrical signals are emitted, as well as the characteristics of the electrical signals provided to the signal delivery devices 110. In some embodiments, input is collected via the external stimulator or trial modulator and can be used by the clinician to help determine what parameters to vary. In a typical process, the practitioner uses a cable assembly 120 to temporarily connect the trial modulator 105 to the signal delivery device 110. The practitioner can test the efficacy of the signal delivery devices 110 in an initial position. The practitioner can then disconnect the cable assembly 120 (e.g., at a connector 122), reposition the signal delivery devices 110 and reapply the electrical signals. This process can be performed iteratively until the practitioner obtains the desired position for the signal delivery devices 110. Optionally, the practitioner may move the partially implanted signal delivery devices 110 without disconnecting the cable assembly 120. Furthermore, in some embodiments, the iterative process of repositioning the signal delivery devices 110 and/or varying the therapy parameters may not be performed.

The signal generator 101, the lead extension 102, the trial modulator 105 and/or the connector 122 can each include a receiving element 109. Accordingly, the receiving elements 109 can be patient implantable elements, or the receiving elements 109 can be integral with an external patient treatment element, device or component (e.g., the trial modulator 105 and/or the connector 122). The receiving elements 109 can be configured to facilitate a simple coupling and decoupling procedure between the signal delivery devices 110, the lead extension 102, the pulse generator 101, the trial modulator 105 and/or the connector 122. The receiving elements 109 can be, at least generally, similar in structure and function to those described in U.S. Patent Application Publication No. 2011/0071593, incorporated by reference herein in its entirety.

After the signal delivery elements 110 are implanted, the patient 190 can receive therapy via signals generated by the trial modulator 105, generally, for a limited period of time. During this time, the patient wears the cable assembly 120 and the trial modulator 105 outside the body. Assuming the trial therapy is effective or shows the promise of being effective, the practitioner then replaces the trial modulator 105 with the implanted signal generator 101 and programs the signal generator 101 with therapy programs selected based on the experience gained during the trial period. Optionally, the practitioner can also replace the signal delivery elements 110. Once the implantable signal generator 101 has been positioned within the patient 190, the therapy programs provided by the signal generator 101 can still be updated remotely via a wireless physician's programmer (e.g., a physician's laptop, a physician's remote or remote device, etc.) 117 and/or a wireless patient programmer 106 (e.g., a patient's laptop, patient's remote or remote device, etc.). Generally, the patient 190 has control over fewer parameters than does the practitioner. For example, the capability of the patient programmer 106 may be limited to starting and/or stopping the signal generator 101, and/or adjusting the signal amplitude. The patient programmer 106 may be configured to accept pain relief input, as well as other variables such as medication use.

In some embodiments, the present technology includes receiving patient feedback via a sensor that is indicative of, or otherwise corresponds to, the patient's response to the signal. Feedback includes, but is not limited to, motor, sensory, and verbal feedback. In response to the patient feedback, one or more signal parameters can be adjusted, such as frequency, pulse width, amplitude or delivery location.

FIG. 1B is a cross-sectional illustration of the spinal cord 191 and an adjacent vertebra 195 (based generally on information from Crossman and Neary, “Neuroanatomy,” 1995 (published by Churchill Livingstone)), along with multiple leads 111 (shown as leads 111a-111e) implanted at representative locations. For purposes of illustration, multiple leads 111 are shown in FIG. 1B implanted in a single patient. In addition, for purposes of illustration, the leads 111 are shown as elongated leads, however, leads 111 can be paddle leads. In actual use, any given patient will likely receive fewer than all the leads 111 shown in FIG. 1B.

The spinal cord 191 is situated within a vertebral foramen 188 between a ventrally located ventral body 196 and a dorsally located transverse process 198 and spinous process 197. Arrows V and D identify the ventral and dorsal directions, respectively. The spinal cord 191 itself is located within the dura mater 199 which also surrounds portions of the nerves exiting the spinal cord 191, including the ventral roots 192, dorsal roots 193 and dorsal root ganglia 194. The dorsal roots 193 enter the spinal cord 191 at the dorsal root entry zone 187 and communicate with dorsal horn neurons located at the dorsal horn 186. In some embodiments, the first and second leads 111a, 111b are positioned just off the spinal cord midline 189 (e.g., about 1 mm offset) in opposing lateral directions so that the two leads 111a, 111b are spaced apart from each other by about 2 mm, as discussed above. In some embodiments, a lead, or pairs of leads, can be positioned at other locations, e.g., toward the outer edge of the dorsal root entry zone 187 as shown by a third lead 111c, or at the dorsal root ganglia 194, as shown by a fourth lead 111d, or approximately at the spinal cord midline 189, as shown by a fifth lead 111e.

In any of the foregoing embodiments, it is important that the signal delivery device 110, and in particular, the therapy or electrical contacts of the device, be placed at a target location that is expected (e.g., by a practitioner) to produce efficacious results in the patient when the device 110 is activated. The following sections describe techniques and systems for simplifying the process of placing contacts via one or more leads 111 to deliver neural modulation signals to the patient.

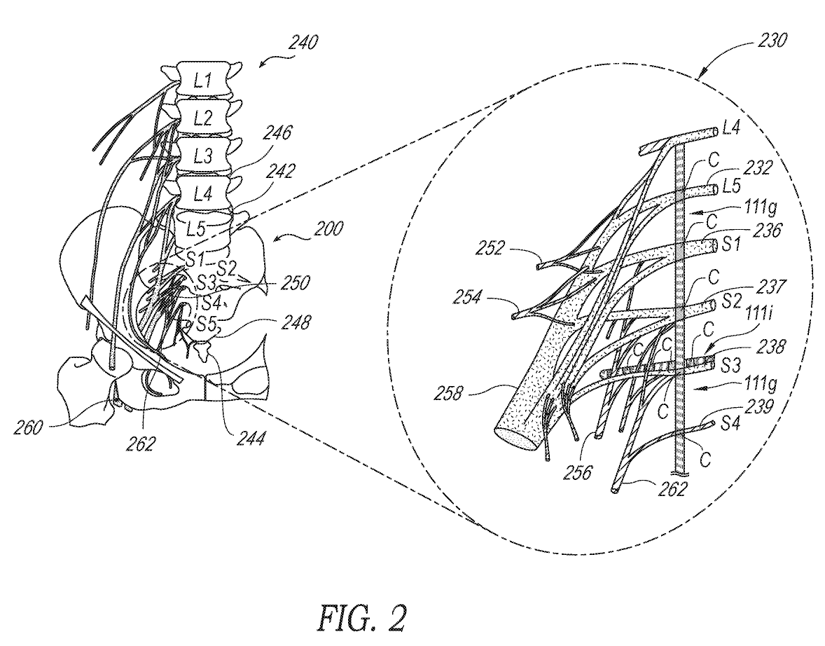

FIG. 2 is an illustration of a sacral region adjacent to the base of the patient's spine 240, and the sacral plexus 250, along with multiple signal delivery devices 111 (shown as signal delivery devices 111g and 111i) implanted at representative locations. For purposes of illustration, multiple signal delivery devices, 111g and 111i are shown in FIG. 2, implanted in a single patient. These signal delivery devices 111g and 111i can have one or more contacts C that can be positioned at or near one or more target locations. In actual use, any given patient may receive fewer than all the signal delivery devices 111g and 111i shown in FIG. 2; the signal delivery devices 111g and 111i may include fewer contacts or more contacts C than are illustrated in FIG. 2, and/or one or more contacts C may be positioned at each target location.

The sacral region (e.g., sacrum) 200 is located adjacent to the base of the spine 240 between the fifth segment of the lumbar region (L5) 242 and the coccyx 244. The sacrum 200 is a generally triangular-shaped bone having five segments (S1-S5) which are generally fused together and end with S5 248. The sacral region 200 includes a plurality of nerves, referred to as the sacral plexus 250, extending from the fourth lumbar vertebrae (L4) 246 through the fourth segment of the sacral region (S4). More specifically, and as shown in the expanded view 230, the sacral plexus 250 includes sacral spinal nerve S1-sacral spinal nerve S4, as well as extensions of the lumbar spinal nerves L4 and L5. The nerves within the sacral plexus 250 are referred to as the superior gluteal nerve 252 (e.g., at L4-S1), the inferior gluteal nerve 254 (e.g., at L5-S2), and the posterior femoral nerve 256 (e.g., at S1-S3). The nerves of the sacral plexus 250 converge near the greater sciatic foramen 258 which has branches that extend into the sciatic nerve 260 (e.g., at L4-S3), the pudendal nerve 262 (e.g., pudendal plexus at S2-S4), and others.

In some embodiments, a signal delivery device 111 can be delivered to the patient's sacral region such that one or more contacts (C) are positioned at or near one or more target locations. For example, a first signal delivery device 111g can be positioned within, at, or approximately at, the patient's L5 lumbar nerve root 232, within, at, or approximately at the patient's S1 sacral nerve root 236, within, at, or approximately at the patient's S2 sacral nerve root 237, within, at, or approximately at the patient's S3 sacral nerve root 238, and/or within, at, or approximately at the patient's S4 sacral nerve root 239. For another example, a second signal delivery device 111i can be positioned along the patient's S3 sacral nerve root 238. Either a transforaminal placement technique, a retrograde technique or a sacral hiatus placement technique can be used to access one or more of the sacral nerve roots and position one or more signal delivery devices (e.g., leads) 111 within, at, or near one or more of the patient's sacral nerve roots. For example, using the transforaminal placement technique, the lead (or leads) 111i can be placed along a portion of a length of the S3 nerve root 238. In another example, using the sacral hiatus placement technique, the lead (or leads) 111g can be positioned transverse relative to one or more sacral nerve roots, such as S1 236-S4 239. It is believed that high frequency modulation at or near one or more of the sacral nerve roots 232, 236, 237, 238, and 239 can produce effective relief from pelvic pain and relief from one or more gastrourogenital conditions, without paresthesia, without adverse sensory or motor effects, and/or in a manner that persists after the modulation ceases.

Without being bound by the following theory, or any other theories, it is expected that the therapy signals act to reduce and/or treat pelvic pain and reduce and/or treat gastrourogenital conditions via one, two, three, or any combination of these three mechanisms: (1) by reducing neural transmissions entering the sacral nerve(s) at the sacral nerve root(s); (2) by reducing neural activity at the sacral nerves themselves; and/or (3) modulating the patient's sympathetic (hypogastric) or parasympathetic (pelvic, pudendal) nervous system. The presently disclosed therapy is expected to produce pain reduction without the side effects generally associated with standard SCS therapies, as discussed further in U.S. Pat. No. 8,170,675, incorporated herein by reference. These and other advantages associated with embodiments of the presently-disclosed technology are described further below.

In some embodiments, the lead is positioned proximate to one or more of the patient's sacral nerves. After positioning, the therapeutic electrical signal is delivered to one or more of the sacral nerves thereby addressing one or more pelvic indications. These pelvic indications are discussed throughout the disclosure and include, but are not limited to, pelvic pain, OAB, one or more symptoms associated with OAB, and/or fecal incontinence.

Clinical evidence also indicates beneficial outcomes for patients having urogenital distress (including OAB, symptoms associated with OAB, and/or fecal incontinence) following placement of a lead at an epidural location proximate to their spinal cord. Accordingly, OAB, symptoms associated with OAB, fecal incontinence, and other indications can be treated by positioning the lead at one or more epidural locations proximate to the spinal cord, such as from T8 to T12. These other indications include but are not limited to, back pain and/or leg pain.

While embodiments of the present technology may create some effect on normal motor and/or sensory signals, the effect is below a level that the patient can reliably detect intrinsically (e.g., without the aid of external assistance) via instruments or other devices. Accordingly, the patient's levels of motor signaling and other sensory signaling (other than signaling associated with the target pelvic pain and gastrourogenital condition) can be maintained at pre-treatment levels. For example, the patient can experience a significant pelvic pain reduction and reduction in one or more gastrourogenital conditions that are largely independent of the patient's movement and position. In particular, the patient can assume a variety of positions, consume various amounts of food and liquid, and/or undertake a variety of movements associated with activities of daily living and/or other activities without the need to adjust the parameters in accordance with which the therapy is applied to the patient (e.g., the signal amplitude). This result can greatly simplify the patient's life and reduce the effort required by the patient to experience pelvic pain relief and a reduction in the gastrourogenital condition (or corresponding symptoms) while engaging in a variety of activities. This result can also provide an improved lifestyle for patients who experience pelvic pain and a gastrourogenital condition during sleep.

In addition, patients can choose from a limited number of programs (e.g., two or three) each with a different amplitude and/or other signal delivery parameter to address some or all of the patient's pelvic pain and gastrourogenital condition. In some embodiments, the patient activates one program before sleeping and another after waking, or the patient activates one program before sleeping, a second program after waking, and a third program before engaging in particular activities that would otherwise cause pelvic pain and trigger, enhance, or otherwise exacerbate the gastrourogenital condition. This reduced set of patient options can greatly simplify the patient's ability to easily manage pelvic pain and gastrourogenital condition, without reducing (and in fact, increasing) the circumstances under which the therapy effectively addresses pelvic pain and gastrourogenital condition. In some embodiments which include multiple programs, the patient's workload can be further reduced by automatically detecting a change in patient circumstance and automatically identifying and delivering the appropriate therapy regimen. Additional details of such techniques and associated systems are disclosed in U.S. Pat. No. 8,355,797, incorporated herein by reference.

Several aspects of the technology are embodied in computing devices (e.g., programmed/programmable pulse generators), controllers and/or other devices. The computing devices on/in which the described technology can be implemented may include one or more central processing units, memory, input devices (e.g., input ports), output devices (e.g., display devices), storage devices and network devices (e.g., network interfaces). The memory and storage devices are computer-readable media that may store instructions that implement the technology. In some embodiments, the computer readable media are tangible media. In some embodiments, the data structures and message structures may be stored or transmitted via an intangible data transmission medium, such as a signal on a communications link. Various suitable communications links may be used, including but not limited to, a local area network and/or a wide-area network.

### 3.0 Addressing Pelvic Pain and Gastrourogenital Conditions

The present technology provides methods and devices for treating and/or reducing pelvic pain, treating and/or reducing a gastrourogenital condition, or both. Methods and systems for treating pain and a gastrourogenital condition by applying high frequency modulation signals to sacral neural populations (e.g., sacral neural populations in the sacral nerves) are discussed immediately below.

“Treating” or “treatment,” as used herein with regard to a condition, refers to preventing the condition, reducing, or eliminating symptoms associated with the condition; generating a complete or partial regression of the condition; or some combination thereof. “Preventing” or “prevention,” as used herein with regard to a condition, refers to total or partial prevention of the condition or symptoms associated with the condition. “Reduce” or “reducing,” as used herein with regard to a condition, disease, disorder, and/or symptom thereof, refers to ameliorating an effect that the condition, disease, disorder, and/or symptom thereof has on a patient; or some combination thereof.

In some embodiments, therapeutic modulation signals are directed generally to the patient's sacral region. More specifically, the therapeutic modulation signals are directed, generally, to the patient's sacral spinal nerves S1-S4. For example, the modulation signals can be directed to S3 in the patient's sacral region to treat pelvic pain, a gastrourogenital condition, and/or pain and a gastrourogenital condition. In another example, the modulation signals can be directed to S2 and/or S4 in the patient's sacral region to treat pelvic pain. In some embodiments, the modulation signals may be directed to other neurological structures and/or target neural populations.

In some embodiments, methods are provided for treating pelvic pain and a gastrourogenital condition by applying electrical stimulation, with the therapy signal parameters disclosed herein, to the sacral region, an organ, and/or another target tissue. The organ and/or target tissue can be an organ of the patient's gastrourogenital system. For example, the target organ includes the bladder, the ureter, the urethra, the uterus, the prostate, the colon, one or more sphincters of the colon, the rectum, the vagina, or the kidney, and the target tissue includes tissues associated with any of the target organs, such as supporting muscular tissue and fascia. The gastrourogenital condition may be a condition listed in Table 1, which provides various sacral targets for applying electrical stimulation in the treatment of each condition. For example, the gastrourogenital condition may be overactive bladder (OAB), urinary incontinence, fecal incontinence, sexual dysfunction, erectile dysfunction, nocturia, urine retention, frequent bladder voiding, frequent urge to void bladder, pelvic and/or abdominal pain, epididymitis, foumier gangrene, genital neoplasms, hematocele, hemospermia, infertility, penile diseases, prostatic diseases, reproductive tract infections, sexually transmitted diseases, spermatic cord torsion, spermatocele, testicular diseases, genital tuberculosis, pelvic floor disorders, renal tuberculosis, bladder exstrophy, cryptorchidism, epispadias, fraser syndrome, fused kidney, hypospadias, multicystic dysplastic kidney, nephritis, retrocaval ureter, solitary kidney, urinary fistula, kidney diseases, ureteral diseases, uretheral disease, urinary bladder diseases, urinary tract infections, urination disorders, urolithiasis, adnexal diseases, endometriosis, gynatresia, reproductive tract infections, uterine diseases, vaginal diseases, vulvar diseases, and/or urolithiasis. In some embodiments, the patient's pain corresponds to and/or is caused by one or more of the conditions listed in Table 1. In some embodiments, the patient can experience pain within, at, and/or near one or more organs and/or tissues listed in Table 1, corresponding to one or more conditions listed in Table 1, and/or affected by one or more conditions listed in Table 1. In some embodiments, the patient's pain corresponds to an inflammatory condition, a physical abnormality, and/or a physical deformity. Treatment may be carried out by applying electrical stimulation to any of the targets listed, or to a combination thereof. The list of targets is not exhaustive, meaning that there may be one or more additional targets for each condition.

TABLE 1
Gastrourogenital Conditions

| Indication | Sacral target | Other targets |
|---|---|---|
| OAB | S3 | Pelvic nerve, hypogastric nerve, |
| | | pudendal nerve, femoral nerve, tibial |
| | | nerve, sciatic nerve, dorsal penile |
| | | nerve, and/or clitoral nerve |
| Urinary incontinence | S1-S5 (e.g., S3) | Pelvic nerve, hypogastric nerve, |
| | | pudendal nerve, femoral nerve, tibial |
| | | nerve, sciatic nerve, dorsal penile |
| | | nerve, and/or clitoral nerve |
| Fecal incontinence | S1-S5 (e.g., S3) | Pelvic nerve, hypogastric nerve, |
| | | pudendal nerve, femoral nerve, tibial |
| | | nerve, sciatic nerve, dorsal penile |
| | | nerve, and/or clitoral nerve |
| Sexual dysfunction | S1-S5 (e.g., S3) | Pelvic nerve, hypogastric nerve, |
| | | pudendal nerve, femoral nerve, tibial |
| | | nerve, sciatic nerve, dorsal penile |
| | | nerve, and/or clitoral nerve |
| Erectile dysfunction | S1-S5 (e.g., S3) | Pelvic nerve, hypogastric nerve, |
| | | pudendal nerve, femoral nerve, tibial |
| | | nerve, sciatic nerve, dorsal penile |
| | | nerve, and/or clitoral nerve |
| Urinary retention | S1-S5 (e.g., S3) | Pelvic nerve, hypogastric nerve, |
| | | pudendal nerve, femoral nerve, tibial |
| | | nerve, sciatic nerve, dorsal penile |
| | | nerve, and/or clitoral nerve |
| Nocturia | S1-S5 (e.g., S3) | Pelvic nerve, hypogastric nerve, |
| | | pudendal nerve, femoral nerve, tibial |
| | | nerve, sciatic nerve, dorsal penile |
| | | nerve, and/or clitoral nerve |
| Frequent bladder voiding | S1-S5 (e.g., S3) | Pelvic nerve, hypogastric nerve, |
| | | pudendal nerve, femoral nerve, tibial |
| | | nerve, sciatic nerve, dorsal penile |
| | | nerve, and/or clitoral nerve |
| Frequent urge to void bladder | S1-S5 (e.g., S3) | Pelvic nerve, hypogastric nerve, |
| | | pudendal nerve, femoral nerve, tibial |
| | | nerve, sciatic nerve, dorsal penile |
| | | nerve, and/or clitoral nerve |
| Infertility | S1-S5 (e.g., S3) | |
| Penile diseases | S1-S5 (e.g., S3) | |
| Prostatic diseases | S1-S5 (e.g., S3) | |
| Testicular diseases | S1-S5 (e.g., S3) | |
| Pelvic floor disorders | S1-S5 (e.g., S3) | |
| Kidney diseases | S1-S5 (e.g., S3) | |
| Ureteral diseases | S1-S5 (e.g., S3) | |
| Urethral diseases | S1-S5 (e.g., S3) | |
| Urinary bladder diseases | S1-S5 (e.g., S3) | |
| Adnexal diseases | S1-S5 (e.g., S3) | |
| Urination disorders | S1-S5 (e.g., S3) | |
| Uterine diseases | S1-S5 (e.g., S3) | |
| Vaginal diseases | S1-S5 (e.g., S3) | |
| Vulvar diseases | S1-S5 (e.g., S3) | |
| Urinary fistula | S1-S5 (e.g., S3) | |
| Urinary tract infections | S1-S5 (e.g., S3) | |
| Urolithiasis | S1-S5 (e.g., S3) | |
| Gynatresia | S1-S5 (e.g., S3) | |
| Bowel dysfunction | S1-S5 (e.g., S3) | |
| Genital neoplasms | S1-S5 (e.g., S3) | |
| Hematocele | S1-S5 (e.g., S3) | |
| Pelvic pain | S1-S5 (e.g., S3) | |
| Pain from epididymitis | S1-S5 (e.g., S3) | |
| Pain from nephritis | S1-S5 (e.g., S3) | |
| Pain from endometriosis | S1-S5 (e.g., S3) | |
| Pain from spermatic cord torsion | S1-S5 (e.g., S3) | |

In some embodiments, methods are provided for determining whether the patient's OAB, or one or more corresponding symptoms, has been reduced or otherwise treated by applying electrical stimulation, with the therapy signal parameters disclosed herein, to the sacral region (e.g., S1-S5), an organ, and/or another target tissue. The methods may include evaluating the patient with one or more measures of success which may be one or more measures of success listed in Table 2. For example, Table 2 provides various measures of success (e.g., success parameters) for determining if applying electrical stimulation to a patient having OAB caused a reduction in the patient's OAB symptoms. The list of measures of success is not exhaustive, meaning that there may be one or more additional, fewer, and/or different success parameters.

TABLE 2
Overactive Bladder - Measures of Success

| Success Parameter | Indication of OAB | Indication of Success |
|---|---|---|
| Voids per day | greater than 8 voids per day. | Decrease by at least about 50% |
| | | in voids per day or a return to a |
| | | normal number of voids per day |
| | | (e.g., 8 or fewer voids per day). |
| Nocturia | Any amount of voiding at night which | Decrease by at least about 50% |
| | causes the patient to awaken. | in the number of nocturia |
| | | incidents. |
| Urge (UUI), Urinary | Urge and leaking (UI) rated on 5-point | Decrease by at least about 50% |
| Incontinence/Leaking | scale (0- no urge, 1-can postpone | in the urge score. |
| (UI) | voiding with no worry of leaks, 2- could | This can be measured using a |
| | postpone voiding for a short while, 3- | voiding diary, pad changes |
| | could not postpone, rushed to toilet, 4- | (e.g., for patients where leaking |
| | leaked before arriving at toilet). | is continual) or a combination |
| | | thereof. |
| Fecal incontinence | Measured using a questionnaire having | Decrease by at least about 50% |
| (FI) | 5 question which address leaks with | in the FI score. |
| | solid stool, leaks with liquid stool, urge, | |
| | underwear changes, and the effect of | |
| | the patient's symptoms on the patient's | |
| | lifestyle. | |
| Pelvic and/or | VAS scale (measured in cm) and pain | Decrease by at least about 50% |
| abdominal pain | map. | in VAS. |

In some embodiments, electrical stimulation is performed with at least a portion of the therapy signal at a frequency in a frequency range between about 1.2 kHz and about 100 kHz; between about 1.5 kHz and about 100 kHz, between about 1.5 kHz and about 50 kHz; between about 3 kHz and about 20 kHz; between about 1.2 kHz and about 25 kHz; between about 1.5 kHz and about 25 kHz; between about 1.2 kHz and about 50 kHz; between about 1.5 kHz and about 50 kHz; between about 3 kHz and about 15 kHz; or between about 5 kHz and about 15 kHz; or at frequencies of about 5 kHz, about 6 kHz, about 7 kHz, about 8 kHz, about 9 kHz, about 10 kHz, about 11 kHz, about 12 kHz, about 25 kHz, or about 50 kHz; and in some embodiments, surprisingly effective results have been found when treating certain medical conditions with frequencies between 5 kHz and 15 kHz, and in some embodiments, at 10 kHz. (Unless otherwise specified, the term “about” is intended to represent +/−10%, or, in the case of a range, a range as would be understood as reasonably equivalent by one of ordinary skill in the art.)

In some embodiments, the electrical stimulation may be applied with at least a portion of the therapy signal at amplitudes within amplitude ranges of about 0.1 mA to about 20 mA; about 0.5 mA to about 10 mA; about 0.5 mA to about 7 mA; about 0.5 mA to about 5 mA; about 0.5 mA to about 4 mA; about 0.5 mA to about 2.5 mA; and in some embodiments, surprisingly effective results have been found when treating certain medical conditions with amplitudes below 7 mA.

In some embodiments, the electrical stimulation may be applied with at least a portion of the therapy signal having a pulse width within a pulse width range of from about 10 microseconds to about 333 microseconds; from about 10 microseconds to about 166 microseconds; from about 25 microseconds to about 166 microseconds; from about 25 microseconds to about 100 microseconds; from about 30 microseconds to about 100 microseconds; from about 33 microseconds to about 100 microseconds; from about 50 microseconds to about 166 microseconds; and in some embodiments, surprisingly effective results have been found when treating certain medical conditions with pulse widths from about 25 microseconds to about 100 microseconds; and from about 30 microseconds to about 40 microseconds.

The therapy signal may be delivered to a patient having pelvic pain and a gastrourogenital condition at a frequency in a frequency range of 1.2 kHz to 100 kHz, such as from about 1.2 kHz to about 25 kHz, from about 1.5 kHz to about 25 kHz, from about 1.2 kHz to about 50 kHz, from about 1.5 kHz to about 50 kHz, about 25 kHz, or about 50 kHz, a pulse width in a pulse width range of 10 microseconds to 333 microseconds and an amplitude in an amplitude range of 0.1 mA to 20 mA in some embodiments in accordance with the present technology. In addition, the therapy signal can be applied at a duty cycle of 5% to 75%, and can be applied to locations within a patient's sacral region to treat and/or reduce pelvic pain, and one or more gastrourogenital conditions, including but not limited to OAB. In some embodiments, a therapy signal having a pulse width is applied to the sacral region at a pulse width in a pulse width range of 10 microseconds to 333 microseconds at any of a variety of suitable frequencies (within or outside the range of 1.5 kHz to 100 kHz) to treat pelvic pain and OAB.

Additionally, aspects of the therapy provided to the patient may be varied while still obtaining beneficial results. In some embodiments, the location of the lead body (and in particular, the lead body electrodes or contacts) can be varied throughout and/or across the target location(s) described above. For example, the lead body can be a percutaneous lead that can be temporarily, and optionally repeatedly, positioned within the patient's body, such as subcutaneously at or near the patient's tibia. As another example, the lead body can be an external lead body, such as a fully external lead body, positioned on a portion of the patient's foot, ankle, shin, calf, knee, thigh, or other suitable location to obtain at least some of the beneficial results described herein. In some embodiments, the signal can be applied to one or more of the patient's peripheral nerves, including but not limited to, the patient's pelvic nerve, hypogastric nerve, pudendal nerve, tibial nerve, sciatic nerve, dorsal penile nerve, and/or clitoral nerve. Other characteristics of the applied signal can also be varied. In some embodiments, (1) the amplitude of the applied signal can be ramped up and/or down, and/or (2) the amplitude can be increased or set at an initial level to establish a therapeutic effect, and then reduced to a lower level to save power without forsaking efficacy, as is disclosed in U.S. Patent Publication No. 2009/0204173, incorporated herein by reference, or (3) both. The signal amplitude may refer to the electrical current level (e.g., for current-controlled systems), or to the electrical voltage level (e.g., for voltage-controlled systems). The specific values selected for the foregoing parameters may vary from patient to patient and/or from indication to indication and/or on the basis of the selected electrical stimulation location, such as the sacral region. In addition, the methodology may make use of other parameters, in addition to, or in lieu of, those described above, to monitor and/or control patient therapy. For example, in cases for which the pulse generator includes a constant voltage arrangement rather than a constant current arrangement, the current values described above may be replaced with corresponding voltage values.

In some embodiments, the parameters, in accordance with which the pulse generator provides signals, can be modulated during portions of the therapy regimen. For example, the frequency, amplitude, pulse width, and/or signal delivery location can be modulated in accordance with a preset program patient and/or physician inputs, and/or in a random or pseudorandom manner. Such parameter variations can be used to address a number of potential clinical situations, including changes in the patient's perception of one or more symptoms associated with the condition being treated, changes in the preferred target neural population, and/or patient accommodation or habituation.

Patients can receive multiple signals in accordance with some embodiments, such as two or more signals, each with different signal delivery parameters. For example, the signals can be interleaved with each other, such as the patient can receive 5 kHz pulses interleaved with 10 kHz pulses. In some embodiments, patients can receive sequential “packets” of pulses at different frequencies, with each packet having a duration of less than one second, several seconds, several minutes, or longer depending upon the particular patient and indication.

In some embodiments, electrical stimulation may be administered on a pre-determined schedule or on an as-needed basis. Administration may continue for a pre-determined amount of time, or it may continue indefinitely until a specific therapeutic benchmark is reached (for example, until an acceptable reduction in one or more symptoms). In some embodiments, electrical stimulation may be administered one or more times per day, one or more times per week, once a week, once a month, or once every several months. Administration frequency may also change over the course of treatment. For example, a patient may receive less frequent administrations over the course of treatment as certain therapeutic benchmarks are met. The duration of each administration (e.g., the actual time during which a patient is receiving electrical stimulation) may remain constant throughout the course of treatment, or it may vary depending on factors such as patient health, internal pathophysiological measures, or symptom severity. In some embodiments, the duration of each administration may range from 1 to 4 hours, 4 to 12 hours, 12 to 24 hours, 1 day to 4 days, or 4 days or greater.

### 5.0 Representative Modulation Locations and Indications

Application of electrical stimulation can be carried out using suitable devices and programming modules specifically programmed to carry out any of the methods described herein. In some embodiments, electrical stimulation is applied to the sacral region, an organ, and/or another target tissue using a device such as those described herein. For example, the device can comprise a lead, wherein the lead in turn comprises an electrode. In some embodiments, administration of electrical stimulation comprises a positioning step (e.g., placing the lead such that an electrode is in proximity to the sacral region, an organ, and/or another target tissue) and a stimulation step (e.g., transmitting an electrical signal (i.e., therapy signal) through the electrode).

Electrical stimulation may be applied directly to the sacral region, an organ, and/or another target tissue, or it may be applied in close proximity to the sacral region, an organ, and/or another target tissue (i.e., close enough for the sacral region, the sacral region, an organ, and/or another target tissue to receive the electrical signal). In some embodiments, electrical stimulation is applied to the sacral region. For example, the electrical stimulation is applied to other neural tissue such as sacral nerves, sacral nerve roots, and peripheral nerves corresponding to the sacral region. For some conditions, electrical stimulation may be applied to a single target tissue or organ. For other conditions, electrical stimulation may be applied to the sacral region, multiple organs, and/or multiple other target tissues. For example, where the condition includes pelvic pain and a gastrourogenital condition, stimulation may be applied to the S3 sacral nerve a target tissue corresponding to S3, an organ corresponding to S3, or a combination thereof. In accordance with the present technology, electrical stimulation parameters may be configured so as to not result in the patient experiencing paresthesia.

A variety of suitable devices for administering an electrical signal to the sacral region, an organ, and/or another target tissue are described in greater detail above in Section 2.0 and may also be taught in the references incorporated by reference herein. Other examples of devices for administering an electrical signal that can treat pain are disclosed in U.S. Pat. Nos. 8,694,108 and 8,355,797, both of which are incorporated herein by reference in their entireties, respectively. In some embodiments, a device that is used for applying an electrical signal to the spinal cord may be repurposed with or without modifications to administer an electrical signal to another target tissue or organ (e.g., a sacral region, a cortical, sub-cortical, intra-cortical, or peripheral target). As such, any of the herein described systems, sub-systems, and/or sub-components serve as means for performing any of the herein described methods.

In some embodiments, the electrical signal can be applied at a particular vertebral level, such as those described herein in accordance with the present technology. In some embodiments, the vertebral level corresponds to the organ of interest. For example, the modulation signal can be applied to S3 to address gastrourogenital conditions. In this example, application of the modulation signal to S3 also treats pelvic pain. In another example, the modulation signal can be applied to S2 and/or S4 to address pelvic pain. In this example, the modulation signal applied to S2 and/or S4 may not treat or reduce OAB, or a symptom corresponding to OAB. Further details of particular vertebral levels and associated organs are described in U.S. Pat. No. 8,170,675, previously incorporated herein by reference.

Many of the embodiments described above were described in the context of treating pelvic pain and gastrourogenital conditions with modulation signals applied to the sacral region, such as S3. For example, modulation may be applied to the sacral region and more particularly, the cauda equina (“horse tail” region) at which the sacral nerves enter the sacrum. Pelvic pain and OAB represent example indications that are expected to be treatable with modulation applied at this location. In some embodiments, modulation signals having parameters (e.g., frequency, pulse width, amplitude, and/or duty cycle) generally similar to those described above can be applied to other patient locations to address other indications.

The methods disclosed herein include and encompass, in addition to methods of making and using the disclosed devices and systems, methods of instructing others to make and use the disclosed devices and systems. For example, a method in accordance with a particular embodiment includes reducing a patient's pelvic pain and treating one or more gastrourogenital conditions by applying an electrical signal to the patient's sacral region with the electrical signal having a frequency in a range of from about 1.5 kHz to about 100 kHz, such as from about 1.2 kHz to about 25 kHz, from about 1.5 kHz to about 25 kHz, from about 1.2 kHz to about 50 kHz, from about 1.5 kHz to about 50 kHz, about 25 kHz, or about 50 kHz. A method in accordance with another embodiment includes instructing or directing such a method. Accordingly, any and all methods of use and manufacture disclosed herein also fully disclose and enable corresponding methods of instructing such methods of use and manufacture.

One feature of several of the embodiments described above is that the therapy signal can be applied to a limited region to address both the patient's pain and the patient's gastrourogenital condition(s). For example, one or more signal delivery contacts (e.g., electrodes) can be positioned at the patient's sacral region to address both pain and gastrourogenital condition(s). An advantage of this arrangement is that it can simplify the process of addressing these multiple patient indications, compared with approaches that require stimulation at multiple regions or locations.

From the foregoing, it will be appreciated that some embodiments of the disclosure have been described herein for purposes of illustration, but that various modifications may be made without deviating from the disclosure. For example, the specific parameter ranges, and indications described above may be different in further embodiments, and/or the target location may be different. In some embodiments, as described above, the signal amplitude applied to the patient can be constant. In other embodiments, the amplitude can vary in a preselected manner (e.g., via ramping up/down) and/or cycling among multiple amplitudes. The signal delivery elements can have an epidural location, as discussed above with regard to FIG. 1B, and in other embodiments, can have a sacral or peripheral location, as discussed above with regard to FIG. 2. As described above, signals having the foregoing characteristics are expected to provide therapeutic benefits for patients having pelvic pain and one or more gastrourogenital conditions when stimulation is applied at S3, and it is believed that this range can extend from about L4 to about S5. In some embodiments, the present technology can be used to address one or more pain indications, such as those described in the references incorporated by reference, besides and/or in addition to pelvic pain.

### 6.0 Additional Embodiments

The methods, systems, and devices described above may be used to deliver a number of suitable therapies (e.g., paresthesia-based therapies and/or paresthesia-free therapies). Examples of such therapies and associated methods, systems, and devices are described in U.S. Patent Publication Nos. 2009/0204173 and 2010/0274314, the respective disclosures of which are herein incorporated by reference in their entireties.

### EXAMPLES

The following examples are provided to further illustrate embodiments of the present technology and are not to be interpreted as limiting the scope of the present technology. To the extent that certain embodiments or features thereof are mentioned, it is merely for purposes of illustration and, unless otherwise specified, is not intended to limit the present technology. One skilled in the art may develop equivalent means without the exercise of inventive capacity and without departing from the scope of the present technology. It will be understood that many variations can be made in the procedures herein described while still remaining within the bounds of the present technology. Such variations are intended to be included within the scope of the present technology. As such, embodiments of the presently disclosed technology are described in the following representative examples.

### Example 1: Overactive Bladder (OAB) Feasibility Study

An OAB feasibility study was designed to obtain short-term evidence of safety & effectiveness of the devices, systems, and methods of the present technology (e.g., Nevro's Senza® System) in patients having symptoms of OAB. The OAB Feasibility Study was a first non-pain indication, first sacral lead placement, single U.S. center, non-blinded, prospective, single arm study, and enrolled eight patients.

Inclusion criteria were as follows: (1) clinically diagnosed with OAB (including diagnoses urge, leak, and high frequency); (2) refractory to conservative therapy and refractory to a minimum of one anticholinergics or antimuscarinics (or unable to tolerate these medications due to side effects or because the patient has a contraindication to these medications) with a documented history of six months or longer; (3) an appropriate candidate for treatment with neuromodulation; (4) at least 22 years of age at the time of enrollment; (5) an appropriate candidate for the electrode implant procedures; and (6) understands written and spoken English, capable of patientive evaluation, willing and capable of giving informed consent, willing and able to comply with study-related requirements, procedures, and visits, adequate cognitive ability to participate in the study as determined by the investigator.

Exclusion criteria were as follows: (1) an untreated urinary tract infection; (2) currently taking an anticholinergic or antimuscarinic medication (must have stopped taking these medications at least ten days prior to entering the study and must remain off these medications throughout the duration of the study); (3) primary stress or mixed incontinence where the stress component supersedes urge incontinence; (4) a medical condition(s) that would interfere with implantation of the electrodes, any study procedures or would confound the evaluation of study endpoints as determined by the Investigator; (5) has used botulinum toxin within the prior six months to treat OAB (use of botulinum toxin greater than six months from enrollment is acceptable); (6) a current diagnosis of a neurological disease that could affect bladder function as determined by the investigator; (7) a current diagnosis or condition such as a coagulation disorder, bleeding diathesis, platelet dysfunction, progressive peripheral vascular disease or uncontrolled diabetes mellitus that presents excess risk for performing the procedure as determined clinically by the investigator; (8) prior use of a neuromodulation device or a sacral nerve stimulation device; (9) an existing drug pump and/or another active implantable device that would be contraindicated for use with the Senza System, based on device labeling; (10) have metastatic malignant disease, active local malignant disease, or a history of bladder cancer, even if they are in remission with no evidence of disease; (11) a history of benign prostatic hyperplasia (BPH); (12) a BMI of ≥45; (13) a life expectancy of less than one year; (14) an active systemic infection or a local infection at the anticipated surgical entry sites; (15) pregnant or nursing; (16) be immunocompromised; (17) be allergic to or have shown prior hypersensitivity to any materials of the Senza® system which come in contact with the body; and (18) concomitantly participating in another clinical study.

The outcomes of the OAB Feasibility Study were (1) effectiveness as measured by VAS, pain mapping, sexual function, fecal incontinence, OAB symptom score and diary, medication, and satisfaction, (2) safety as measured by neurologic status and adverse events, and (3) success as measured by at least a 50% reduction in OAB symptoms from baseline at the end of trial (EoT). These outcome measures were determined by (1) each patient's OAB Symptom Score (OABSS) consisting of four questions to capture daytime frequency, nighttime frequency, urgency and incontinence, (2) an OAB Diary used for seven days during baseline assessments and then used again daily for the entire duration of the sacral nerve stimulation (7-14 days) to capture daytime frequency of voiding, nighttime frequency of voiding, and incontinence episodes, (3) the patient's VAS score obtained from a scaled psychometric instrument to report pain severity where patients will score the severity of pain on a 10 cm line, with zero indicating no pain and ten indicating the worst pain imaginable, (4) a Pain Map where patients shaded a diagram of the abdominal and pelvic regions of any pain they may be experiencing in these areas, (5) a Fecal Incontinence Questionnaire/Revised Fecal Incontinence Assessment (RFIA) which consisted of five questions and measured on a scale of never to experience daily, the frequency fecal incontinence and lifestyle effects, (6) a Sexual Function Questionnaire (SFQ), (7) each patient's medication usage which included recording all prescription medication and over the counter pain medication usage, (8) a Patient Satisfaction Questionnaire assessed using a five-point scale where responses ranged from “very satisfied” to “very dissatisfied” (summary results were presented, (9) assessing patients for adverse events starting at enrollment and continuing through study completion, and (10) assessing patients for neurological outcomes using examinations which include assessment of motor, sensory and reflex functions.

For the VAS, each patient's baseline pain score was compared to the score at the follow-up visit. Mean changes from baseline were calculated for the entire cohort. The percentage of patients who achieved ≥50% (pelvic) pain relief was also calculated based on changes from baseline.

The SFQ was administered in two parts. The first questionnaire was completed by the patients at baseline and consisted of five questions about patient sexual functioning. The second questionnaire consisted of three questions that mirror the baseline questionnaire and were administered during the follow-up visit.

Following enrollment in the OAB Feasibility Study, patients were evaluated for entry criteria and baseline characteristic measurements were taken (Table 3). Baseline measurements included, (1) a seven-day diary, (2) OAB symptom score questionnaires, (3) fecal incontinence, (4) sexual health, (5) pain mapping by location and severity of pelvic pain, (6) medical and surgical history, and (7) neurological assessment.

Nevro's Senza® system leads were implanted in each patient and positioned on top of the sacral nerves, rather than at the dorsal horn and/or column. Positioning was determined using radiographic images, as well as sensory and/or motor testing performed by modulating the stimulation to the sensory range of the patient. Some patients may also have had a motor response (e.g., plantar flexion of greater toe) from this stimulation that verified correct lead location. In patients that did not report any sensation or motor response, lead location was left to the physician's discretion. Medication intake and any adverse events were also observed. Following lead placement, neurological assessments were performed and one or more adjustments for OAB relief were programmed into the Sensa® system based on patient feedback.

High frequency stimulation was delivered to the patients as therapeutic electrical signals. Leads were removed from each patient after 7 to 14 days of stimulation, and the patients were assessed for EoT metrics including urgency, frequency, incontinence, non-obstructive retention, and nocturia as measured by an OAB questionnaire, fecal incontinence questionnaire, pain assessment (VAS) and pain map, patient satisfaction questionnaire, medication usage, sensory/motor assessment to assess lead location, radiographic images, and adverse events and a neurological assessment.

TABLE 3
Baseline Measurements

| | |
|---|---|
| No. pts 8 | No. primary prestudy diagnosis |
| No. female (%) 8 (100%) | Overactive bladder 8 (100%) |
| No. male (%) 0 (0) | Pelvic pain 4 (50%) |
| No. race (%): | Mean_SD age at trial 59.4_4.45 |
| Caucasian 8 (100%) | Mean_SD yrs since diagnosis 14.1_10.70 |
| African American 0 (0) | OAB interventions prior to trial |
| Asian/Caucasian 0 (0) | Oral drugs/pharmaceutical 8 (100%) |
| American Indian or Alaska native/Caucasian 0 (0) | Lifestyle changes 7 (87.5%) |
| American Indian or Alaska native 0 (0) | Behavioral therapy 6 (75%) |
| Native Hawaiian or other Pacific Islander 0 (0) | Physical Therapy 1 (12.5%) |
| Other 0 (0) | Botox 1 (12.5%) |
| No. primary prestudy symptoms (%): | Hydrodistention 1 (12.5%) |
| Urge 8 (100%) | OAB medications tried prior to trial (%): |
| Urge-Frequency 8 (100%) | Depression 7 (87.5%) |
| Nocturia 8 (100%) | Anxiety 3 (37.5%) |
| Urge-Incontinence 7 (87.5%) | Interstitial Cystitis 2 (25%) |
| Pain 4 (50%) | Insomnia 2 (25%) |
| Fecal-Incontinence (12.5%) | Mean_SD baseline leaks/day 5.10_4.67 (8) |
| No. primary prestudy etiology (%): | Mean_SD No. baseline voids/day 10.53_2.88 (8) |
| Uterine fibroids 2 (25%) | Mean_SD urgency voids 2.42_0.88 (8) |
| Interstitial cystitis 1 (12.5%) | |
| Pudendal neuralgia 1 (12.5%) | |
| Sexual abuse 1 (12.5%) | |

Follow-up studies were performed 7 to 14 days after the EoT metrics were obtained. These included an overactive bladder questionnaire, a fecal incontinence questionnaire, a sexual health questionnaire, a pain assessment (VAS) and pain map, a patient satisfaction questionnaire, medication usage, and a neurological assessment and adverse event screen.

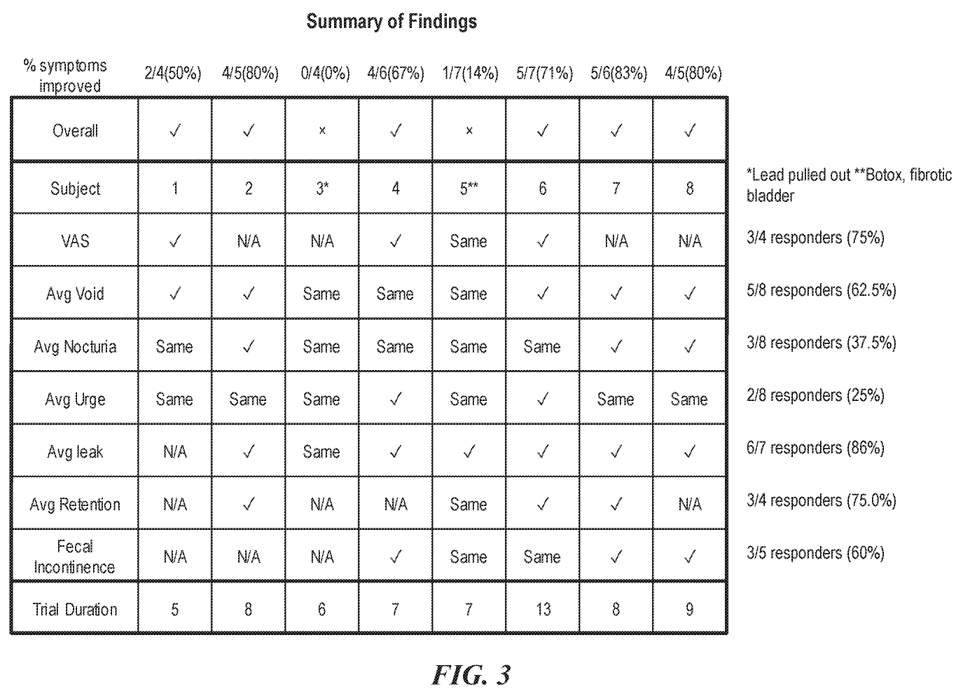

FIG. 3 summarizes the outcomes of the OAB Feasibility Study. As shown in FIG. 3, 75% of responders had improved VAS scores, 62.5% of responders had reduced void frequency (average void), 86% of responders had reduced leak frequency, 75% of responders had improved retention, and 60% of responders had reduced fecal incontinence. Rates for nocturia and urge were lower than expected in view of the higher responder rates for the other metrics. It is thought that the rates for urge were falsely low because the patientive scale used to measure changes in urge was not well-defined and lacked an anchor.

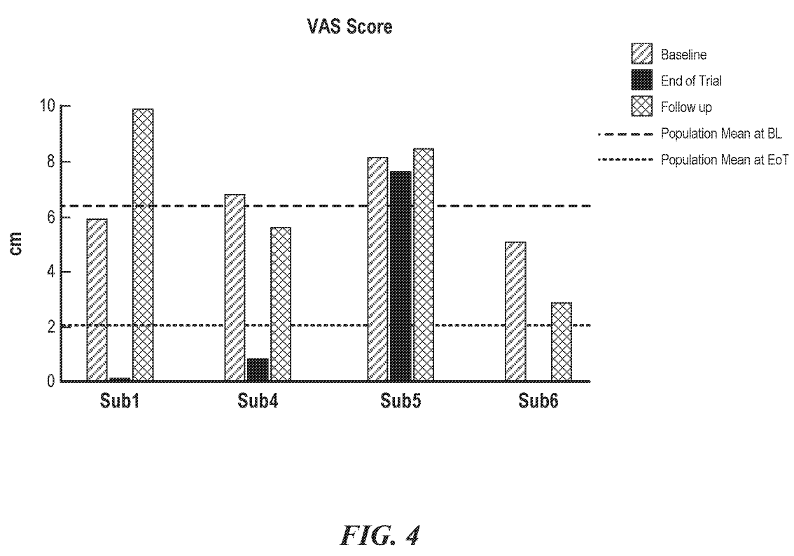

FIG. 4 illustrates VAS scores for four patients who reported having pain during the entry criteria evaluation and baseline characteristic measurement. All four patients reported improvements in pain during the OAB Feasibility Study. Aside from patient 5 (Sub5) whose lead migrated following placement, three of the four patients who reported improvements in pain had markedly improved pain relief during the OAB Feasibility Study and at EoT. Their pain returned during the follow-up period.

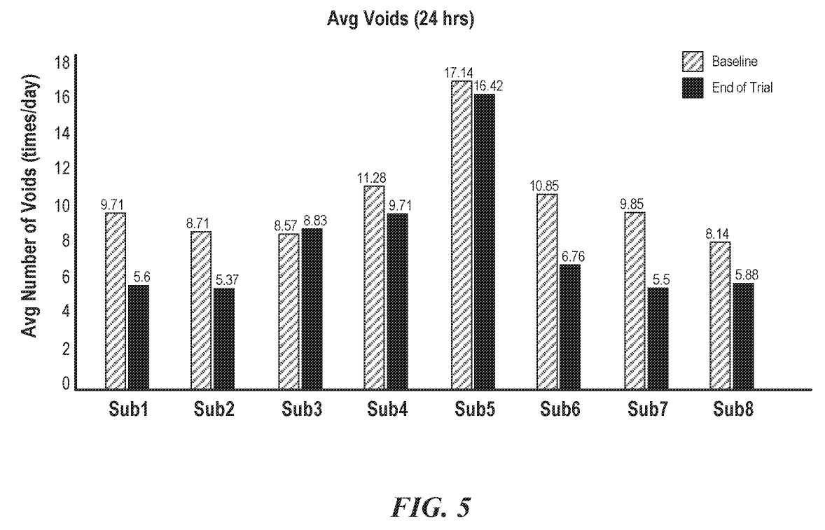

FIG. 5 illustrates the average number of voids within a 24-hour period for patients enrolled in the OAB Feasibility Study. Five of the eight patients (62.5%) enrolled in the study showed improvements in the number of voids per day. For example, for these five patients, the number of voids were reduced at EoT from baseline. The normal average voids per day in a patient not having OAB is eight or less and, while the OAB Feasibility Study defined a responder as having a 50% decrease in symptoms at EoT compared to baseline, fewer than eight voids per day is within a normal range. All five patients having improved voids were therefore normal at EoT.

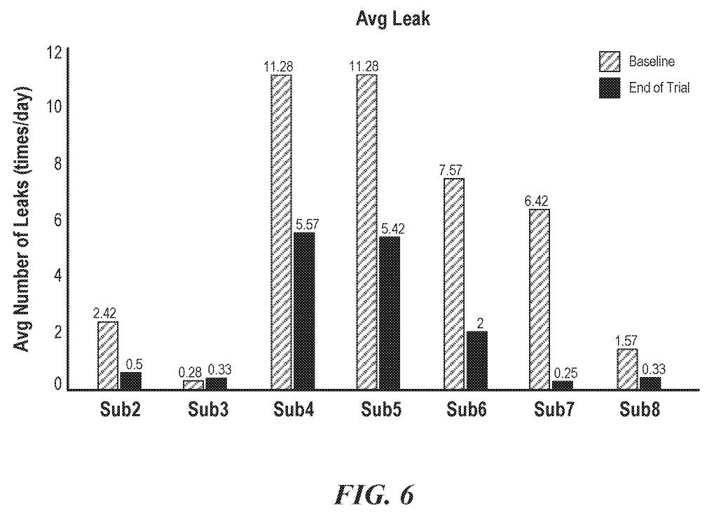

FIG. 6 illustrates the average number of leaks within a 24-hour period for patients enrolled in the OAB Feasibility Study. Six of seven patients (86%) who reported having leaks when entry criteria and baseline characteristic measurements were taken responded to treatment, showing greater than a 50% improvement in the average number of leaks per day at EoT compared to baseline.

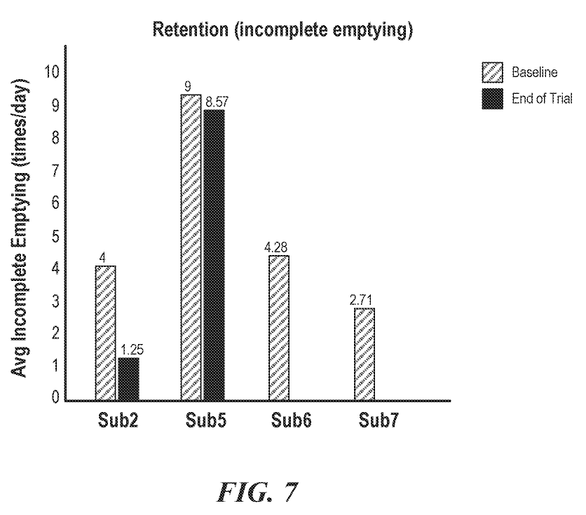

FIG. 7 illustrates the average number of times a patient's bladder failed to completely empty (e.g., retention) within a 24-hour period. A patientive assessment was selected for the OAB Feasibility Study. Three of four patients (75%) who reported failure to completely empty their bladders when entry criteria and baseline characteristic measurements were taken responded to treatment, showing greater than a 50% improvement in the average number of leaks per day at EoT compared to baseline. Unlike void and leak, retention is a patientive assessment. Follow-on studies may use catheterization as an alternative measurement for assessing retention to avoid any confounding information obtained as a result of retention being a subjective assessment.

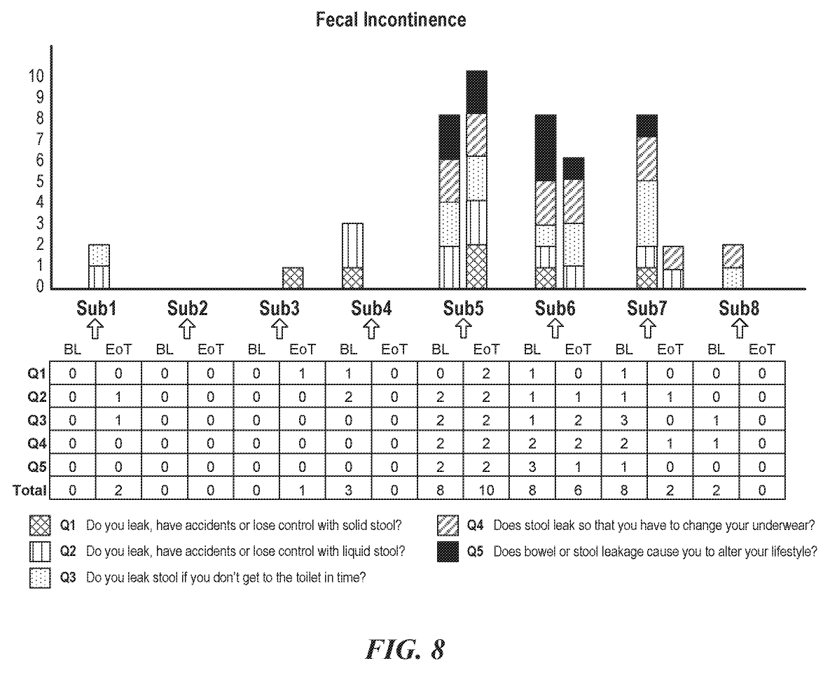

FIG. 8 illustrates changes in fecal incontinence reported by patients enrolled in the OAB Feasibility Study between baseline and EoT. Three of five patients (60%) who reported experiencing at least some degree of fecal incontinence when entry criteria and baseline characteristic measurements were taken responded to treatment, showing greater than a 50% improvement in the average number of leaks per day at EoT compared to baseline. Similar to retention, fecal incontinence is at least a partially subjective assessment, and in this study, the questions posed to patients were not well anchored.

Unlike low frequency stimulation, which results in a motor response, high frequency stimulation of the OAB Feasibility Study did not result in any patients experiencing an evoked response sensation which can manifest as tightening of one or more of the patient's scrotum, vagina, or thigh or movement of the buttock muscles (“bellows”).

Certain aspects of the technology described in the context of particular embodiments may be combined or eliminated in other embodiments. In addition, while advantages associated with some embodiments of the technology have been described in the context of some embodiments, other embodiments may also exhibit such advantages, and not all embodiments need necessarily exhibit such advantages to all within the scope of the present technology. Accordingly, the disclosure and associated technology can encompass other embodiments not expressly shown or described herein.

As used herein, the term “and/or” when used in the phrase “a and/or b” refers to “a” alone, to “b” alone, and to both “a and b.” A similar manner of interpretation applies to the term “and/or” when used in a list of more than two terms.

To the extent any materials incorporated by reference herein conflict with the present disclosure, the present disclosure controls.

1. A method for treating a patient, comprising: Directing an electrical therapy signal to the patient's sacral region via an implantable signal delivery device to reduce or eliminate pelvic pain and/or one or more gastrourogenital conditions in the patient, wherein the electrical signal has a frequency in a frequency range of from 1.2 kHz to 100 kHz and does not produce paresthesia in the patient.

2. The method of claim 1 wherein the gastrourogenital condition includes a bowel dysfunction.

3. The method of claim 1 wherein the gastrourogenital condition includes a bladder dysfunction.

4. The method of claim 1 wherein the gastrourogenital condition includes a sexual dysfunction.

5. The method of claim 2 wherein the bowel dysfunction includes fecal incontinence.

6. The method of claim 3 wherein the bladder dysfunction includes at least one of overactive bladder, urinary incontinence, urine retention, nocturia, frequent bladder voiding or urge to void the bladder.

7. The method of claim 4 wherein the sexual dysfunction includes erectile dysfunction.

8. The method of claim 1, further comprising using patient feedback to position the signal delivery device within the patient.

9. The method of claim 8 wherein using patient feedback includes delivering the electrical therapy signal via the implantable signal delivery device at a frequency of less than 1.5 kHz, and receiving sensory feedback from the patient.

10. The method of claim 8 wherein using patient feedback includes delivering the electrical therapy signal via the implantable signal delivery device at a frequency of less than 1.5 kHz, and receiving motor feedback from the patient.

11. The method of claim 8 wherein using patient feedback includes delivering the electrical therapy signal via the implantable signal delivery device at a frequency of less than 1.5 kHz, and receiving verbal feedback from the patient.

12. The method of claim 1 wherein a pulse width of the electrical therapy signal is in a pulse width range from 10 microseconds to 333 microseconds.

13. The method of claim 12 wherein the pulse width of the electrical therapy signal is in a pulse width range from about 30 microseconds to about 35 microseconds.

14. The method of claim 1 wherein an amplitude of the electrical therapy signal has a non-zero value of about 10 milliamps or less.

15. The method of claim 14 wherein the amplitude of the electrical therapy signal has a non-zero value of 5 milliamp or less.

16. The method of claim 15 wherein the amplitude of the electrical therapy signal has a non-zero value of 1 milliamp or less.

17. The method of claim 16 wherein the amplitude of the electrical therapy signal has a non-zero value of 0.5 milliamp or less.

18. The method of claim 17 wherein the amplitude of the electrical therapy signal has a non-zero value of 0.3 milliamp or less.

19. The method of claim 18 wherein the amplitude of the electrical therapy signal has a non-zero value of 0.1 milliamp or less.

20. The method of claim 1 wherein directing the electrical therapy signal to the patient's sacral region includes directing the electrical therapy signal to one or more sacral nerves.

21. The method of claim 1 wherein directing the electrical therapy signal to the patient's sacral region further comprises directing the electrical therapy signal to a foramen of one or more sacral nerves.

22. The method of claim 20 wherein the one or more sacral nerves includes one or more sacral nerve roots.

23. The method of claim 22 wherein the one or more sacral nerves includes at least one sacral nerve corresponding to S1, S2, S3, S4, or S5.

24. The method of claim 24 wherein the one or more sacral nerves is the sacral nerve corresponding to S3.

25. The method of claim 1 wherein the sacral region includes a sacral location that is chosen so as to treat one or more of the medical conditions listed in Table 1 above.

26. A method for treating a patient having one or more symptoms corresponding to overactive bladder, comprising: directing an electrical therapy signal to a sacral nerve of the patient via an implantable signal delivery device to reduce or eliminate one or more symptoms corresponding to overactive bladder in the patient, wherein the electrical signal has a frequency in a frequency range of from 1.2 kHz to 100 kHz.

27. The method of claim 26 wherein the sacral nerve is a sacral nerve corresponding to S3.

28. The method of claim 27 wherein the sacral nerve corresponding to S3 is a sacral nerve root.

29. The method of claim 26 wherein the patient has pelvic pain.

30. The method of claim 26, further comprising using patient feedback to position the signal delivery device within the patient.

31. The method of claim 30 wherein using patient feedback includes delivering an electrical therapy signal via the implantable signal delivery device at a frequency of less than 1.2 kHz, and receiving sensory feedback from the patient.

32. The method of claim 30 wherein using patient feedback includes delivering an electrical therapy signal via the implantable signal delivery device at a frequency of less than 1.2 kHz, and receiving motor feedback from the patient.

33. The method of claim 26 wherein using patient feedback includes delivering the electrical therapy signal via the implantable signal delivery device at a frequency of less than 1.5 kHz, and receiving verbal feedback from the patient.

34. The method of claim 26 wherein a pulse width of the electrical therapy signal is in a pulse width range from 10 microseconds to 333 microseconds.

35. The method of claim 34 wherein the pulse width of the electrical therapy signal is in a pulse width range from about 30 microseconds to about 35 microseconds.

36. The method of claim 26 wherein an amplitude of the electrical therapy signal has a non-zero value of about 10 milliamps or less.

37. The method of claim 36 wherein the amplitude of the electrical therapy signal has a non-zero value of 1 milliamp or less.

38. The method of claim 37 wherein the amplitude of the electrical therapy signal has a non-zero value of 0.5 milliamp or less.

39. The method of claim 38 wherein the amplitude of the electrical therapy signal has a non-zero value of 0.1 milliamp or less.

40. The method of claim 26 wherein directing the electrical therapy signal to the sacral region further comprises directing the electrical therapy signal to a foramen of one or more sacral nerves.

41. The method of claim 26 wherein the patient exhibits one or more measures of success listed in Table 2 above.

42. A method for treating a patient, comprising: directing an electrical therapy signal to the patient's sacral region via an implantable signal delivery device to reduce or eliminate pelvic pain and one or more gastrourogenital conditions in the patient, wherein the electrical signal has a frequency in a frequency range of from 1.2 kHz to 100 kHz.

43. The method of claim 42 wherein the gastrourogenital condition includes a bowel dysfunction or a bladder dysfunction.

44. The method of claim 43 wherein the bowel dysfunction includes fecal incontinence.

45. The method of claim 43 wherein the bladder dysfunction includes at least one of overactive bladder, urinary incontinence, urine retention, nocturia, frequent bladder voiding or urge to void the bladder.

46. The method of claim 42, further comprising using patient feedback to position the signal delivery device within the patient.

47. The method of claim 46 wherein using patient feedback includes delivering the electrical therapy signal via the implantable signal delivery device at a frequency of less than 1.5 kHz, and receiving sensory feedback from the patient, receiving motor feedback from the patient or receiving verbal feedback from the patient.

48. The method of claim 42 wherein a pulse width of the electrical therapy signal is in a pulse width range from 10 microseconds to 333 microseconds.

49. The method of claim 42 wherein an amplitude of the electrical therapy signal has a non-zero value of about 10 milliamps or less.

50. The method of claim 42 wherein directing the electrical therapy signal to the patient's sacral region includes directing the electrical therapy signal to one or more sacral nerves.

51. The method of claim 50 wherein the one or more sacral nerves includes at least one sacral nerve corresponding to S1, S2, S3, S4, or S5.

52. The method of claim 42 wherein the sacral region includes a sacral location that is chosen so as to treat one or more of the medical conditions listed in Table 1 above.
